(19) United States
(12) Patent Application Publication
(10) Pub. No.: US 2025/0134852 A1
(43) Pub. Date: May 1, 2025

(54) QUERCETIN-BASED COMPOSITIONS FOR THE TREATMENT AND PREVENTION OF OCULAR AFFECTIONS

(71) Applicant: Indena S.P.A., Milano (IT)

(72) Inventors: Antonella Riva, Milano (IT); Giovanna Petrangolini, Milano (IT); Pietro Allegrini, Milano (IT); Stefano Togni, Milano (IT); Fabio Mazzolani, Bergamo (IT)

(73) Assignee: Indena S.P.A., Milano (IT)

(21) Appl. No.: 18/835,362

(22) PCT Filed: Feb. 3, 2023
(86) PCT No.: PCT/IB2023/050952

§ 371 Date: Aug. 2, 2024

(30) Foreign Application Priority Data
Feb. 7, 2022 (IT) 102022000002108

(51) Int. Cl. A61K 31/352; A61K 9/00; A61K 9/20; A61P 27/02

(52) U.S. Cl. CPC A61K 31/352; A61K 9/0053; A61K 9/2009; A61K 9/2013; A61K 9/2059; A61P 27/02

(57) ABSTRACT

Compositions comprising a solid dispersion of quercetin are useful in the treatment and/or prevention of ocular affections.

The invention relates to oral composition useful in the treatment and/or prevention of ocular affections.

### BACKGROUND OF THE INVENTION

Flavonoids, or bioflavonoids, are a class di polyphenolic compounds ubiquitous in plant species; therein, such compounds perform various functions such as flower pigmentation, act as UV filters, chemical messengers, and physiological regulators. To date, about five-thousands have been isolated and characterized, subdivisible, for example, in the following subsets: anthocyanidins, chalcones, flavones, flavonols, flavanones, flavanonols, flavanols, flavandiols, isoflavones, isoflavanes, isoflavandiols, cumestanes, pterocarpans, etc.

Quercetin, compound of formula 1 belonging to the flavonols subset, is present, usually in a glycosylated form, in various fruits and vegetables, such as apples, berries, grapefruit, onions, capers, radishes, and their derivatives, as in red wine and tea.

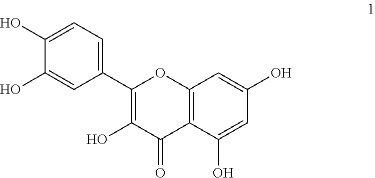 [SMILES: O=c1c(O)c(-c2ccc(O)c(O)c2)oc2cc(O)cc(O)c12]

In vitro, quercetin showed anti-inflammatory, immuno-modulatory, anti-allergic, anti-fibrotic, anti-coagulative, anti-bacterial, anti-atherogenic, anti-hypertensive, and anti-proliferative properties (“Health effects of quercetin: from mechanism to nutraceutical,” Agnes W. Boots, 2006).

Due to the poor bioavailability, caused by water insolubility, quercetin applications in the nutraceutical and pharmaceutical field do not reflect the potential shown in the in vitro tests.

The formulation as a solid dispersion comprising quercetin, phospholipids, and a water-soluble carrier, allows to increase quercetin solubility, thus widening the spectrum of possible applications (WO 2019/016146).

The treatment and/or prevention of ocular affections, among which the ones involving the ocular epithelium, usually provides for the topical application of the medicine; such administration route, even if representing the election administration mode, has some drawbacks: the therapeutical effect depends strictly on the drug ability to penetrate the ocular epithelium, thus precluding such application to highly water insoluble drugs; further, due to the eye anatomy and physiology, only about 1% of the applied dose is effectively absorbed, while the remaining part is discarded by drainage or blinking, tear turnover, tear dilution, tear evaporation, induced lacrimation, enzymatic degradation, or bonding with the tear fluid proteins, etc. (“Sviluppo di inserti oftalmici liofilizzati per la somministrazione di antimicrobici peptidici” [Development of lyophilized ophthalmic inserts for the administration of peptide antimicrobials], Serena Tivegna, 2015). Such drawbacks lead to the need to repeat the medicine application several times during the day, making the adherence to the therapeutic plan difficult to respect by the treated subject. Moreover, the constant eye manipulation could lead to the development of bacterial infections, for example caused by the medicine application without previous hand washing, or damages of traumatic nature, such as rubbing with nails or the end of the medicine applicators.

In WO2013/164512 the in vitro anti-inflammatory and anti-oxidant activity of polyphenolic compounds, such as quercetin and resveratrol, alone or in mixture, on a culture of human corneal epithelium and in vivo, as topic formulation, in a rat animal model of the pathology known as dry eyes is reported.

CN 1110141599 discloses the preparation of an ointment for the treatment of the chronical catarrhal conjunctivitis comprising botanical extracts, in turn comprising quercetin.

US2017258864 discloses a composition comprising at least an extended release component comprising quercetin and at least a fast release component comprising an extract of Perilla frutescens or a constituent therein contained.

In light of the multiple biological properties shown by quercetin, the need to identify new possible pharmaceutical and nutraceutical applications of the formulation thereof as a solid dispersion remains present.

### DESCRIPTION OF THE INVENTION

The Applicant has surprisingly developed a new application of compositions comprising quercetin; such application allows to use quercetin in the treatment and/or prevention of ocular affections.

The present invention relates, therefore, to the use of compositions comprising quercetin for the treatment and/or prevention of ocular affections; particularly of the ocular epithelium; more particularly, the use of compositions comprising quercetin in the form of a solid dispersion; even more particularly, the use by oral route of compositions comprising quercetin in the form of a solid dispersion for the treatment and/or prevention of affections of the ocular epithelium.

Typically, ocular affections involve the iris, cornea, pupil, anterior chamber, crystalline, conjunctiva, sclera; preferably, cornea and conjunctiva.

Typically, ocular affections can be conjunctivitis, allergic conjunctivitis, corneal lacerations with and without edema caused for example by traumas, superficial punctate and linear keratitis, dry eyes, and other dysfunctions, such as blurred vision and eye fatigue, related to prolonged exposure to light sources with a high proportion of blue light, such as computer screens, mobile phones, and tablets.

For sake of clarity, in the present invention the term “ocular epithelium” means the ocular structures consisting of epithelial tissue such as conjunctiva or corneal epithelium, the term “affections” means abnormal conditions of the epithelium deriving from traumas, pathogen agents intervention or immune system excessive reaction; the term “solid dispersion” means a system of at least two components, wherein at least a first component is dispersed in at least a second component and the obtained system is physically uniform. In a first aspect, the invention relates to compositions comprising a quercetin solid dispersion for the treatment and/or prevention of ocular affections. Such solid dispersion comprises quercetin, a phospholipid and, optionally, a water very soluble to freely soluble carrier.

In the present invention the terms “very soluble” and “freely soluble” have the meaning, as from European Pharmacopeia 5.0, set in the Table 1 below:

TABLE 1

| | Approximate solvent volume (mL) for |
|---|---|
| Term | amount (g) of solute, at T = 15-25° C. |
| Very soluble | less than 1 |
| Freely soluble | from 1 to 10 |

For sake of clarity, “very soluble” carrier means that 1 g of said carrier is completely dissolved in less than 1 mL of solvent (water) at a temperature comprised between 15-25° C., while “freely soluble” means that 1 g of carrier is completely dissolved in an amount of solvent (water) comprised between 1-10 mL at a temperature comprised between 15-25° C.

Phospholipids can be selected from soy, sunflower, or egg lecithin, phosphatidylcholine, phosphatidylserine, phosphatidylethanolamine, or mixtures thereof, in those phospholipids the acyl groups, same or different from each other, derive, for example, from palmitic, stearic, oleic, linoleic, linolenic acids.

The weight ratio between quercetin and phospholipids can range from 5:1 to 1:1, preferably from 2:1 to 1:1.

The water very soluble to freely soluble carrier can be selected from the group consisting of mono- and di-saccharides (as saccharose, fructose, maltodextrins); polyalcohols (as mannitol, sorbitol, xylitol); oligo- and polysaccharides (as dextran, pullulan).

The weight ratio between quercetin and carrier can range from 1:5 to 5:1, preferably from 1:2 to 3:1.

Typically, the compositions can further comprise one or more excipients of food or pharmaceutical grade such as disintegrants, lubricants, binders, coating agents, colorants, adsorption enhancers, solubilizing agents, stabilizers, flavors, sweeteners, antiseptics, preservatives, antioxidants, and the like.

An example of a preferred solid dispersion is consisting of: quercetin, sunflower lecithin, and maltodextrins, wherein the weight ratio between quercetin and sunflower lecithin is equal to 1:1, and the weight ratio between quercetin and maltodextrins is 2.2:1.

Typically, the compositions comprising the quercetin solid dispersion are orally administered in the form of chewable tablets, capsules, soft gelatin capsules, hard gelatin capsules, lozenges, chewable lozenges, pills, tablets, orodispersible tablets, granules for oral solutions, syrups, gels, pads, drops, drinks; preferably, tablets.

With particular reference to the first preferred aspect, the invention relates to the use of oral compositions comprising a quercetin solid dispersion for the treatment and/or prevention of ocular affections involving the iris, cornea, pupil, anterior chamber, crystalline, conjunctiva, sclera such as conjunctivitis, allergic conjunctivitis, corneal lacerations with and without edema caused for example by traumas, superficial punctate and linear keratitis, dry eyes.

A second preferred aspect is represented by the combined preparation for concurrent, separate, or sequential use in the treatment and/or prevention of ocular affections of the compositions comprising a solid dispersion of quercetin and a further drug; typically, such drug is selected from anti-allergenics, antibiotics, anti-inflammatoires, pain relievers, antihistamines, or antivirals.

For sake of clarity, the expression “concurrent, separate, or sequential use in the treatment and/or prevention of ocular affections” means that the therapeutic and/or preventive plan of the ocular affections provides for the intake of both the oral composition comprising a quercetin solid dispersion and the further drug, regardless of their respective administration methodologies.

A preferred embodiment is represented by the combined preparation of the oral compositions comprising a quercetin solid dispersion and a topical antibiotic, for the treatment of ocular affections deriving from traumas.

In a study carried out by the Applicant, seven subjects having minor or major lacerations, or inflammations, such as keratitis, caused by traumas of mechanical, thermal, and chemical nature, and described in detail in the Table 2, were treated with a broad-spectrum topical antibiotic and a quercetin oral solid dispersion. For such subjects, after a period of time comprised between 3-7 days, a decrease in pain and a scarring of the damaged tissue were noted regardless of the type of offender. Without being tied to the theory, both the decrease in pain and the fast scarring are surprising effects attributable to the administration of quercetin 1.

Although quercetin is known to have anti-inflammatory properties due to the ability to inhibit cyclooxygenases (COXs) and lipoxygenases (LOXs) (Li Y. et al., Nutrients, 2016, 8, 167), its effect on the reduction of the inflammation at ocular level is surprising: in fact, the presence of a complex system of blood-ocular barrier prevents concentrations of orally administered drugs such to allow the achievement of a therapeutical effect to reach the inside of the eye. The quercetin formulation as a solid dispersion not only allows to increase the quercetin bioavailability, but also to pass through the blood-ocular barrier. Usually, for the treatment of the ocular affections wherein an inflammatory process is implied, topical formulations comprising anti-inflammatory drugs such as corticosteroids or nonsteroidal anti-inflammatory drugs are used; anyway, such treatments can slow down the tissue scarring process, thus prolonging the healing times. The use of quercetin as anti-inflammatory agent, influencing the scarring times, allows a faster scarring of the epithelial tissue.

A further embodiment is represented by the combined preparation of a quercetin solid dispersion and a topical anti-histamine for the treatment and/or prevention of allergic conjunctivitis.

In a study carried out by the Applicant, fifteen subjects were treated for fifteen days with a quercetin solid dispersion, two tablets per die, and an antibiotic, one drop each eye twice a day. The efficacy of the treatment was verified in the subjects before the beginning and at the end of the treatment using the TOSS questionnaire and measuring the tear film breakup time. The TOSS (“Total Ocular Symptom Score”) questionnaire is commonly used by allergists to evaluate ocular symptoms (ocular itch, lacrimation, conjunctival congestion, ocular hyperemia, and photophobia) in subjects suffering from allergic conjunctivitis, who assign to each symptom a value comprised between 0 and 3, according to the symptom severity; as noted from the questionnaire results set in the Table 3, at the end of the study it was observed, in almost all cases, that subjects reported disappearance of symptoms or showed a light symptomatology, regardless of the symptom severity before the treatment.

The tear film breakup time was measured by the NIF-BUT (“Non Invasive First Breakup Time”) and NIVag-BUT (“Non Invasive Average Breakup Time”) tests; the NIF-BUT test indicates the time, expressed in seconds and tenths of a second, that elapses between the opening of the eye following to one more blinking and the instant wherein the first break ever on the tear film appears, or the instant wherein a further blinking of the eyelid happens; the NIVag-BUT test indicates the average time of the tear film breakup calculated averaging the times of all sectors of the disc reflexed on the cornea that actually broke, appearing distorted, over the course of time. The results obtained from the two tests, reported in the Tables 4 and 5, analyzed with a descriptive statistic, demonstrated a significative improvement in the tear film breakup time. In fact, the tear film breakup time undergoes an increase of at least the 65%, 70%, 75%, 80%, 85%, 90%, 95%, 100%, 105%, 110%. Moreover, with particular reference to the results obtained from the NIF-BUT measurement, it is noted that the tear film breakup time passes from a borderline condition to a normal condition.

Although anti-inflammatory and anti-allergic potentials are known for quercetin (Shaik Y. B. et al., J Biol Regul Homeost Agents. 2006, 20 (3-4): 47-52; Chirumbolo S., Inflamm. Allergy-Drug Targets 2010; 9 (4): 263-85), the achievement of such therapeutical effect for the oral intake of quercetin is not expected and due to the aforementioned reasons. Moreover, the positive effect on the tear film not only is advantageous for the reduction of the discomfort in patients suffering from allergic conjunctivitis, but also can be considered advantageous for healthy subjects; for example, healthy subjects wearing contact lenses can lead to dry eyes since, due to external factors such as the central heating, wind, sun, prolonged exposure to light sources with a high proportion of blue light etc., the lenses absorb hydration from the tear film; such dry eyes could need not only the application of tear drops, but could even cause eye irritation and trigger inflammatory phenomena.

A further unexpected effect related to the intake of quercetin in the form of a solid dispersion of the invention is represented by the fact that the condition in the treated subjects not only improved during the two weeks treatment, but also that such positive effect persists for the two following weeks; differently from the topically administered medicines, running out their effect at the end of the treatment, the oral intake of quercetin revealed its efficacy beyond the end of the treatment, thus preventing the recurring of the studied ocular affections.

With particular reference to the second preferred aspect, the invention relates to the use of oral compositions comprising a quercetin solid dispersion in combination with a further drug for the treatment and/or prevention of ocular affections involving the iris, cornea, pupil, anterior chamber, crystalline, conjunctiva, sclera such as conjunctivitis, allergic conjunctivitis, corneal lacerations with and without edema caused for example by traumas, superficial punctate and linear keratitis, dry eyes.

### EXPERIMENTAL

### Materials and Methods

A commercially available quercetin-based solid composition was administered (Quercefit®, Indena S.p.A. Italy) and formulated in the form of tablets for oral administration; said composition comprises quercetin, sunflower lecithin, maltodextrins, and silica.

The antibiotic olopatadine (0.1% solution-Pataday®, Alcon USA) was administered in the form of eye drops. The tear film breakup time was assessed by the NIF-BUT and NIVag-BUT tests.

### Example 1—Verification of the Efficacy of an Oral Composition Comprising Quercetin in the Treatment of Corneal Lesions

To a subject with a corneal lesion, more or less severe, a treatment with a broad-spectrum topical antibiotic, for a number of days related to the type and extent of the damage, in association with the oral intake of a Quercefit® tablet (250 mg/die) for thirty days, was prescribed. Such treatment led to the decrease of the pain associated with the corneal lesion and promoted the scarring of the corneal epithelium in a range comprised between 3-7 days.

In the Table 2 the details of the single treated subjects are set:

TABLE 2

| | Lesion | | | Time of |
|---|---|---|---|---|
| Subject | type | Cause | Diagnosis | healing |
| 1 | Mechanical | Nail rubbing | Large sealed corneal | 7 days |
| | | | laceration with | |
| | | | moderate corneal | |
| | | | edema | |
| 2 | Mechanical | Improper use | Superficial punctate | 3 days |
| | | of contact | keratitis | |
| | | lenses | | |
| 3 | Mechanical | Presence of an | Linear keratitis | 7 days |
| | | external body | | |
| 4 | Mechanical | Presence of an | Large sealed corneal | 7 days |
| | | external body | laceration with | |
| | | | moderate corneal | |
| | | | edema | |
| 5 | Mechanical | Presence of an | Corneal epithelium | 4 days |
| | | external body | laceration | |
| 6 | Thermal | Hot oil splash | Focal punctate | 7 days |
| | | | erosion of the corneal | |
| | | | epithelium and diffuse | |
| | | | conjunctival injection | |
| 7 | Chemical | Sketch of | Corneal epithelium | 3 days |
| | | cleaner | laceration | |

### Example 2—Verification of the Efficacy of an Oral Composition Comprising Quercetin in the Treatment of Allergic Conjunctivitis

Fifteen subjects were treated for a 15 days period with two Quercefit® tablets (500 mg/die) and a drop each eye of olopatadine (0.1% solution) twice a day.

Primary endpoint: changing of the averaged value obtained from the “The Total Ocular Symptom Score (TOSS) questionnaire.”

TABLE 3
Results of TOSS questionnaire

| | Photo- | | Ocular | | | | Conjunctival | | Ocular | |
|---|---|---|---|---|---|---|---|---|---|---|
| | phobia | | itch | | Lacrimation | | congestion | | Hyperemia | |
| Subject | t₀ | t₁₅ | t₀ | t₁₅ | t₀ | t₁₅ | t₀ | t₁₅ | t₀ | t₁₅ |
| 1 | 1 | 0 | 2 | 0 | 2 | 0 | 2 | 0 | 1 | 0 |
| 1 | 1 | 0 | 2 | 0 | 2 | 0 | 2 | 0 | 1 | 0 |
| 2 | 1 | 0 | 2 | 0 | 2 | 0 | 1 | 0 | 1 | 0 |
| 2 | 1 | 0 | 2 | 0 | 2 | 0 | 1 | 0 | 1 | 0 |
| 3 | 1 | 0 | 2 | 0 | 3 | 1 | 1 | 0 | 1 | 0 |
| 3 | 1 | 0 | 3 | 1 | 2 | 0 | 1 | 0 | 1 | 0 |
| 4 | 1 | 0 | 3 | 1 | 1 | 0 | 1 | 0 | 1 | 0 |
| 4 | 1 | 0 | 3 | 0 | 1 | 0 | 1 | 0 | 1 | 0 |
| 5 | 2 | 0 | 3 | 0 | 1 | 0 | 1 | 0 | 1 | 0 |
| 5 | 2 | 0 | 3 | 0 | 1 | 0 | 1 | 0 | 1 | 0 |
| 6 | 2 | 1 | 3 | 0 | 2 | 1 | 1 | 0 | 1 | 0 |
| 6 | 2 | 1 | 3 | 1 | 1 | 1 | 1 | 0 | 1 | 0 |
| 7 | 1 | 0 | 3 | 1 | 2 | 0 | 1 | 0 | 2 | 1 |
| 7 | 1 | 0 | 3 | 1 | 2 | 1 | 1 | 0 | 2 | 1 |
| 8 | 1 | 0 | 2 | 0 | 1 | 1 | 1 | 0 | 1 | 0 |
| 8 | 1 | 0 | 2 | 1 | 1 | 0 | 1 | 0 | 1 | 0 |
| 9 | 1 | 0 | 3 | 0 | 1 | 0 | 2 | 0 | 1 | 0 |
| 9 | 1 | 0 | 3 | 0 | 2 | 0 | 2 | 0 | 1 | 0 |
| 10 | 1 | 0 | 3 | 0 | 2 | 0 | 2 | 0 | 2 | 1 |
| 10 | 1 | 0 | 3 | 0 | 1 | 0 | 2 | 0 | 2 | 1 |
| 11 | 1 | 0 | 3 | 0 | 1 | 0 | 2 | 0 | 2 | 1 |
| 11 | 1 | 0 | 3 | 0 | 2 | 0 | 2 | 0 | 1 | 0 |
| 12 | 1 | 0 | 3 | 0 | 2 | 1 | 2 | 0 | 1 | 0 |
| 12 | 1 | 0 | 3 | 0 | 1 | 0 | 2 | 0 | 1 | 0 |
| 13 | 1 | 0 | 2 | 0 | 1 | 0 | 1 | 0 | 1 | 0 |
| 13 | 1 | 0 | 3 | 0 | 2 | 1 | 1 | 0 | 1 | 0 |
| 14 | 1 | 0 | 3 | 0 | 2 | 0 | 1 | 0 | 2 | 0 |
| 14 | 1 | 0 | 3 | 0 | 2 | 0 | 1 | 0 | 2 | 0 |
| 15 | 1 | 0 | 3 | 0 | 1 | 0 | 1 | 0 | 2 | 0 |
| 15 | 1 | 0 | 3 | 0 | 2 | 0 | 1 | 0 | 2 | 0 |

where 0 = no symptoms; 1 = light symptoms; 2 = moderate symptoms; and 3 = severe symptoms;
t₀ = before treatment, t₁₅ = at the end of the treatment;

Secondary endpoint: improving of the tear film breakup time.

The tear film breakup time is the time, measured in seconds, elapsing between a blink of an eye and the first dry spot appeared in the tear film. In non-invasive measurement techniques, a grid or a concentric circle pattern is projected on the cornea; when the eye starts to dry, the projected image appears as distorted. Usually, the tear film breakup time is classified as follows: normal (TBUT>10 s), borderline (10 s>TBUT>5 s), and low (TBUT<5 s).

NIF-BUT (Non Invasive First Breakup Time)

TABLE 4

| Subject | t₀ | t₁₅ |
|---|---|---|
| 1 | 2.6 | 5.7 |
| 1 | 2.6 | 1.6 |
| 2 | 1.6 | 17 |
| 2 | 9 | 17 |
| 3 | 2.5 | 17 |
| 3 | 17 | 17 |
| 4 | 7.9 | 12.4 |
| 4 | 6.9 | 9.1 |
| 5 | 2.4 | 17 |
| 5 | 1.4 | 17 |
| 6 | 1.6 | 17 |
| 6 | 6.7 | 17 |
| 7 | 6.7 | 8.4 |
| 7 | 5.2 | 17 |
| 8 | 3.1 | 3.9 |
| 8 | 6 | 12 |
| 9 | 1.6 | 5.7 |
| 9 | 8.1 | 3.1 |
| 10 | 4 | 17 |
| 10 | 7.6 | 6 |
| 11 | 5.5 | 4 |
| 11 | 5.5 | 4 |
| 12 | 6 | 13.9 |
| 12 | 4 | 6.2 |
| 13 | 4 | 17 |
| 13 | 7.6 | 6 |
| 14 | 1.6 | 5.7 |
| 14 | 8.1 | 3.1 |
| 15 | 3.1 | 3.9 |
| 15 | 6 | 12 |
| Average | 5.2 | 10.5 |
| Standard deviation | 3.2 | 5.7 |

### NIVag-BUT (Non Invasive Average Breakup Time)

TABLE 5

| Subject | t₀ | t₁₅ |
|---|---|---|
| 1 | 11.9 | 13.3 |
| 1 | 7.5 | 8.4 |
| 2 | 1.6 | 17 |
| 2 | 9 | 17 |
| 3 | 4 | 17 |
| 3 | 17 | 17 |
| 4 | 7.9 | 13.5 |
| 4 | 6.9 | 9.1 |
| 5 | 5.1 | 17 |
| 5 | 4.6 | 17 |
| 6 | 8.6 | 17 |
| 6 | 10.5 | 17 |
| 7 | 6.7 | 8.4 |
| 7 | 5.2 | 17 |
| 8 | 6.6 | 3.9 |
| 8 | 6.7 | 12 |
| 9 | 2.5 | 5.7 |
| 9 | 10.2 | 4.3 |
| 10 | 7.9 | 17 |
| 10 | 7.6 | 6 |
| 11 | 5 | 17 |
| 11 | 7.2 | 4 |
| 12 | 10 | 13.9 |
| 12 | 6 | 9 |
| 13 | 10.2 | 4.3 |
| 13 | 7.9 | 17 |
| 14 | 6.7 | 12 |
| 14 | 2.5 | 5.7 |
| 15 | 5.2 | 17 |
| 15 | 6.6 | 3.9 |
| Average | 7.2 | 11.9 |
| Standard deviation | 3.0 | 5.2 |

1. A method of treating and/or preventing ocular affections in a subject in need thereof, said method comprising
orally administering to said subject compositions comprising a quercetin solid dispersion, a phospholipid, and a water very soluble carrier, said water very soluble carrier having a complete solubility at a temperature comprised between 15-25° C. of 1 g of said carrier in a water volume of 1 mL or less; or in a water freely soluble carrier, said water freely soluble carrier having a complete solubility at a temperature comprised between 15-25° C. of 1 g of said carrier in a water volume comprised between 1 and 10 mL.

2. The method according to claim 1, wherein the phospholipid is selected from soy, sunflower, or egg lecithin, phosphatidylcholine, phosphatidylserine, phosphatidylethanolamine, or mixtures thereof.

3. The method according to claim 1, wherein the water very soluble to freely soluble carrier is selected from mono-, di-, oligo-, and polysaccharides.

4. The method according to claim 1, wherein the ocular affections are conjunctivitis, allergic conjunctivitis, corneal lacerations with and without edema caused by traumas, superficial punctate and linear keratitis, dry eyes, blurred vision, eye fatigue and dysfunctions related to prolonged exposure to light sources with a high proportion of blue light.

5. (canceled)

6. The method according to claim 1 in the form of chewable tablets, capsules, soft gelatin capsules, hard gelatin capsules, lozenges, chewable lozenges, pills, tablets, oro-dispersible tablets, granules for oral solutions, syrups, gels, pads, drops, drinks.

7. The method according to claim 1 and a further drug for concurrent, separate, or sequential administration wherein the further drug is selected from anti-allergenics, antibiotics, anti-inflammatoires, pain relievers, or antivirals.
